(19) United States
(12) Patent Application Publication
(10) Pub. No.: US 2026/0166349 A1
(43) Pub. Date: Jun. 18, 2026

(54) FILTER CONTAINING PHARMACEUTICAL SALT FOR A FACE MASK, BREATHABLE FACE MASK CONTAINING THE FILTER, AND METHOD OF MANUFACTURING

(71) Applicants: Lisa Semerly, Okemos, MI (US); Jacqueline Semerly, Okemos, MI (US)

(72) Inventors: Lisa Semerly, Okemos, MI (US); Jacqueline Semerly, Okemos, MI (US)

(21) Appl. No.: 19/534,274

(22) Filed: Feb. 9, 2026

Related U.S. Application Data
(62) Division of application No. 17/213,801, filed on Mar. 26, 2021, now Pat. No. 12,544,603.
(60) Provisional application No. 63/000,027, filed on Mar. 26, 2020.
(60) Provisional application No. 63/081,626, filed on Sep. 22, 2020.

(51) Int. Cl. A62B 23/02; A41D 13/11; A61M 21/00; A61M 21/02; A62B 7/10; B01D 39/16

(52) U.S. Cl. CPC A62B 23/025; A41D 13/1192; A61M 21/02; A62B 7/10; B01D 39/1623; A61M 2021/0016; B01D 2239/0407; B01D 2239/0442; B01D 2239/045; B01D 2239/0618; B01D 2239/065; B01D 2239/10; B01D 2239/1216; B01D 2239/1241

(57) ABSTRACT

A filter which includes a layer of non-woven material containing particles of pharmaceutical salt and optionally essential oil for use in a face mask, is provided. The particles can be added by bathing, infused by a halogenerator, or added by other methods. The filter can be placed in a pocket on an exterior side of a facial section, or otherwise attached to the facial section of the face mask. The salt filter deactivates the virus or other pathogen and thus effectively limits the transmission of viruses and other pathogens to and from a person wearing the face mask. The facial section can be formed of a mesh material. The face mask, including the salt filter, is preferably breathable, and light-weight, reusable.

This U.S. divisional patent application claims priority to U.S. utility patent application Ser. No. 17/213,801, filed Mar. 26, 2021, which claims priority to U.S. provisional patent application No. 63/000,027, filed Mar. 26, 2020, and U.S. provisional patent application No. 63/081,626, filed Sep. 22, 2020, the entire contents of which are incorporated herein by reference.

### BACKGROUND OF THE INVENTION

### 1. Field of the Invention

This invention relates to a filter for a face mask, a face mask including a filter, and methods of manufacturing the filter and face mask.

### 2. Related Art

Viruses and other pathogens travel and spread through the air; in droplets, such as saliva or phlegm from coughing, sneezing, speaking, or breathing; and on surfaces. Face masks are often used in attempt to prevent transmission of the viruses and other pathogens from one infected person to another. However, conventional surgical face masks are not sufficient in some cases. For example, droplets carrying the virus or other pathogen, such as droplets expelled from coughing, sneezing, speaking, or breathing, can stay on the surface of a conventional mask. Conventional masks do not kill the virus on the surface. Thus, the virus could be transmitted upon contact with the mask. In addition, conventional face masks, although marketed as “breathable”, are difficult to wear for long periods of time and while engaging in physical activities, such as sports.

Air filtering devices, such as N95 respirators, are also used to reduce infection risk associated with respiratory viruses. However, N95 respirators may not be adequate in providing sufficient protection against some airborne pathogens. Many factors are involved in affecting the efficacy of a filter. The first being that the filter may not sufficiently filter out some smaller size airborne particles and pathogens, providing limited protection against these airborne pathogens. For example, as explained by Hyo-Jick Choi, National Institute for Occupational Safety and Health (NIOSH)-certified N95 respirators cannot provide an expected level of protection against 40-50 nm infectious particles including aerosols. Additionally, the efficacy of the filter that may be used in a mask is also dependent upon on how well the mask or shield fits the face. The mask must fit properly to avoid exposure of airborne pathogens from entering the sides of the mask or shield. Adequate training by a professional must take place on how to properly use an N95 mask, and this requirement may limit public use. In addition, the N95 respirators may be difficult to wear for long periods of time of while engaging in activities.

Face masks coated in salt have also been developed in attempt to prevent transmission of viruses on the surface of face masks. See, for example: https://www.businessinsider.com/mask-coated-in-salt-neutralizes-viruses-like-coronavirus-2020-2. As described by Hyo-Jick Choi in this article, when a virus-carrying droplet encounters a mask coated in a salt solution, the droplet absorbs the salt. Once the liquid of the droplet evaporates, all that remains is the virus and crystallized salt which slices through the virus, neutralizing it. This process completes in about the amount of time it takes the water to evaporate.

However, there remains a great need for additional and/or improved methods to limit the transmission of viruses and pathogens. More specifically, there is a need for sustainable, re-usable, and breathable face masks.

### SUMMARY

One aspect of the invention provides a method for making a face mask that not only effectively limits the transmission of viruses and pathogens, but is sustainable, re-usable, light-weight, and more breathable than the prior art face masks. The face mask of the present invention is desirable to individuals engaging in activities which require a face mask for extended periods of time, for example athletes.

According to one embodiment, the method of making the face mask includes infusing particles of 99.99% pure sodium chloride into a filter layer formed of a first material. The infusing step is conducted by a halogenerator. The method further includes disposing the filter layer infused with the particles along a facial section formed of a second material.

According to another embodiment, the method of making the face mask includes bathing a filter layer formed of a first material in a liquid containing particles of 99.99% pure sodium chloride. The method further includes disposing the filter layer containing the particles along a facial section formed of a second material.

Another aspect of the invention provides a method of making a filter for a face mask. The method comprises infusing particles of 99.99% pure sodium chloride into a filter layer formed of a first material. The infusing step is conducted by a halogenerator.

Yet another aspect of the invention provides a face mask including a filter layer and a facial section. The filter layer is formed of a first material and contains particles of 99.99% pure sodium chloride, and the facial section is formed of a second material. The filter layer is disposed along the facial section of the face mask.

Yet another aspect of the invention provides a filter for a face mask. The filter includes a filter layer formed of a first material and containing particles of 99.99% pure sodium chloride.

### BRIEF DESCRIPTION OF THE DRAWINGS

Advantages of the present invention will be readily appreciated, as the same becomes better understood by reference to the following detailed description when considered in connection with the accompanying drawings.

FIG. 1 illustrates a face mask including a filter layer infused with particles of 99.99% pure sodium chloride in a pocket on an exterior side of a facial section according to an example embodiment.

FIG. 2 illustrates the filter layer infused with particles of 99.99% pure sodium chloride, an inner layer, and an outer layer which can be attached together to form a filter for a face mask according to an example embodiment.

FIGS. 3 and 4 illustrate face masks including the filter layer according to other example embodiments.

FIG. 5 illustrates the filter layer infused with particles of 99.99% pure sodium chloride according to another example embodiments.

FIG. 6 illustrates a variety of materials that can be used to form the layers of the filter for the face mask.

FIGS. 7 and 8 show examples of a halogenerator used to form the filter layer infused with particles of 99.99% pure sodium chloride according to example embodiments.

### DETAILED DESCRIPTION OF EXAMPLE EMBODIMENTS

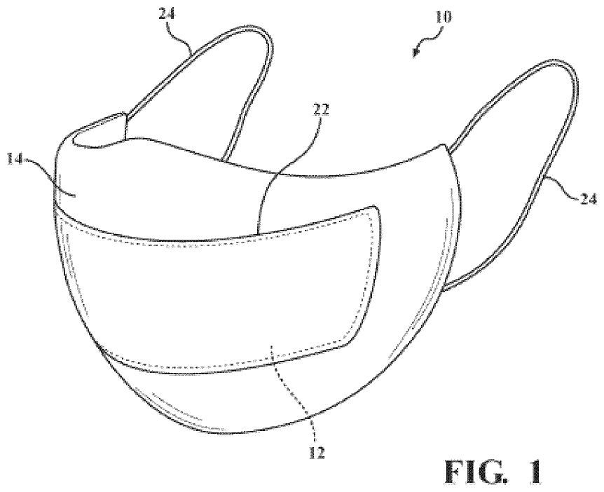

One aspect of the invention provides a method of making a face mask 10 which effectively limits the transmission of viruses and other pathogens, and which is also re-usable, light-weight, and more breathable than the prior art face masks. Due to these characteristics, individuals engaging in physical activities will find the face mask 10 made according to embodiments of the invention desirable to wear for extended periods of time. An example of the face mask 10 is shown in FIG. 1.

The face mask 10 includes a filter layer 12 attached to a facial section 14. The filter layer 12 is formed of a first material, which is preferably a non-woven material but may be a woven material. Examples of non-woven materials include SNS spunbonded non-wovens, Grade 95 meltblown cloth, or activated carbon material. The first material could also be a high efficiency particulate air (HEPA) filter or cotton.

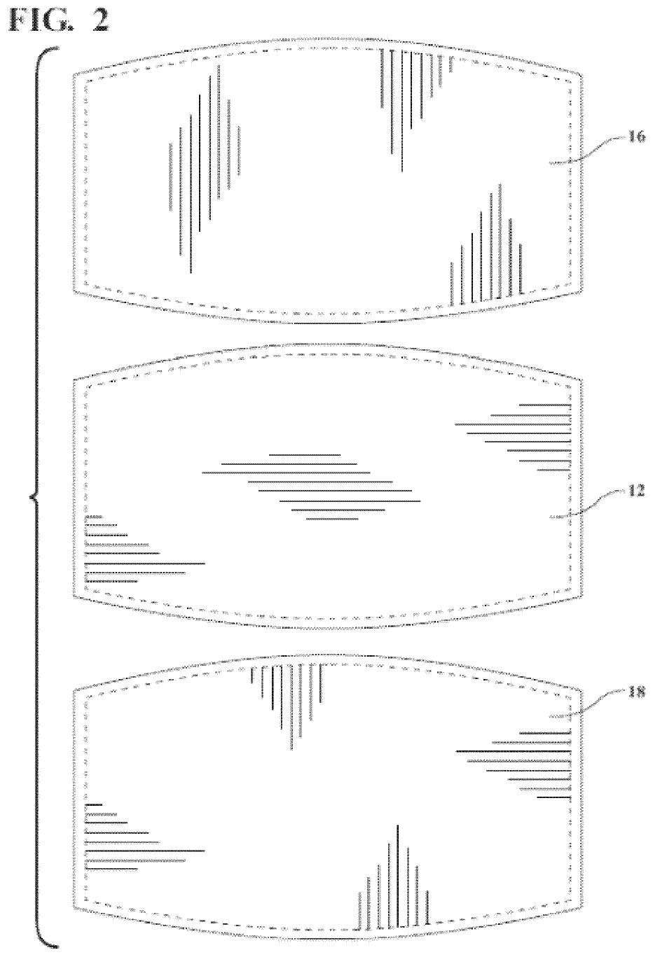

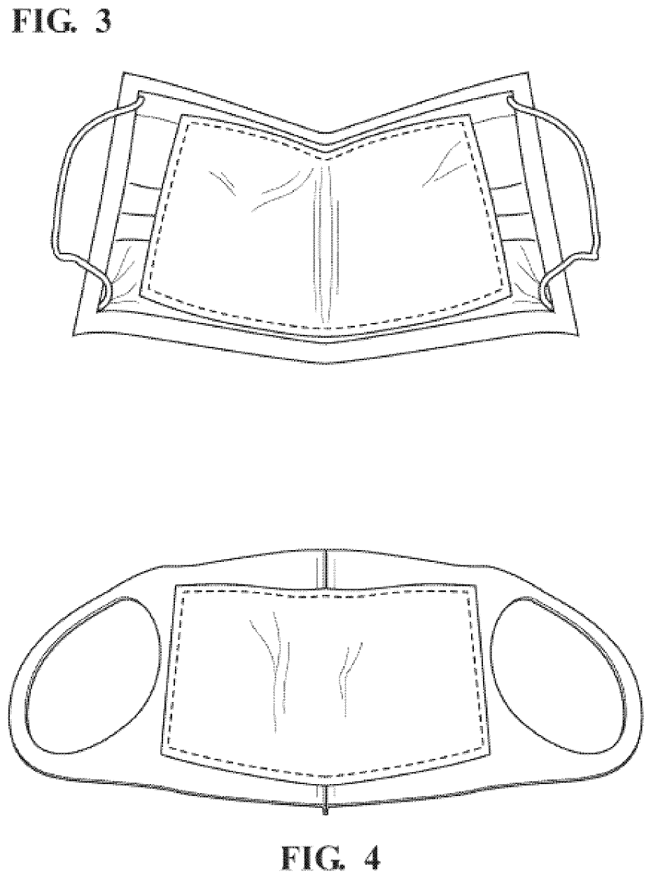

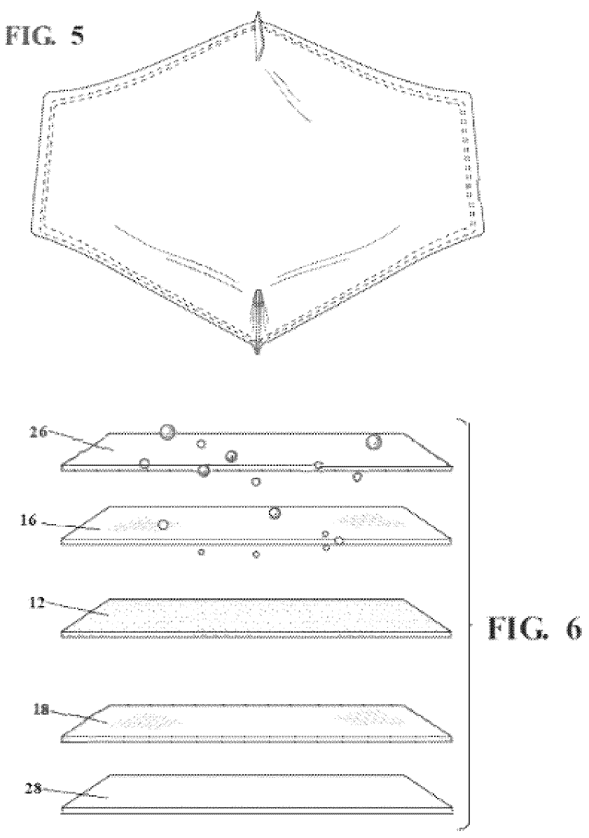

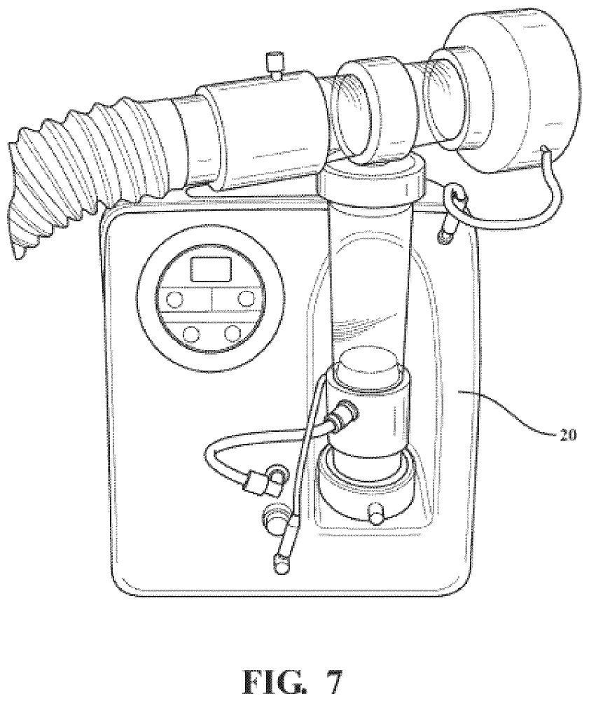

The filter layer 12 can be used on its own, but typically, the filter layer 12 is used with at least one additional layer, preferably three or more layers, more preferably three to five layers. For example, an inner layer 16 formed of an inner material, and an outer layer 18 formed of an outer material can be attached to opposite sides of the filter layer 12 to form a filter. An example of the three layers 12, 16, 18 is shown in FIG. 2. The inner and outer materials can be non-woven, such as SNS spunbonded non-wovens, Grade 95 meltblown filter cloth, or activated carbon material, high efficiency particulate air (HEPA) filter, or woven. The material can be anti-sticking. FIGS. 3 and 4 illustrate the face mask 10 with the filter layer 12 attached to additional layers according to other example embodiments. According to these embodiments, the filter layer 12 has a size of 4.7 inch×3.1 inch (+/−2 inches). FIG. 5 illustrates the filter layer 12 attached to additional layers according to another example embodiment. According to this embodiment, the filter layer 12 is 4.8 inches (+/−1 inch)×3.86 inches (+/−1 inch). FIG. 6 illustrates the filter layer 12 formed of a carbon material and located between two layers formed of Grade 95 meltblown cloth 16, 18. Also in FIG. 6, two layers of the SNS spunbonded non-wovens 16, 18 are disposed on opposite sides of the Grade 95 meltblown cloth 16, 18 (one SNS spunbonded non-woven 16 outward of one Grade 95 meltblown cloth 16, and another SNS spunbonded non-woven 18 outward of the other Grade 95 meltblown cloth 18).

The step of making the filter layer 12 includes infusing particles of at least 99.99% pure sodium chloride (pharmaceutical salt) into the first material. The particles could be 100% pure sodium chloride. The at least 99.99% pure sodium chloride is antimicrobial, antibacterial, and antiviral.

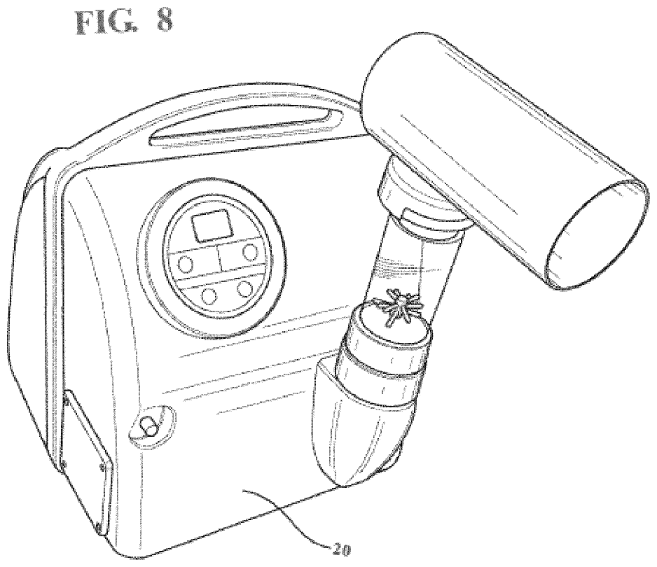

The infusing step is typically conducted by a halogenerator 20 which grinds salt to form the particles and blows the particles of pharmaceutical salt into the filter layer 12. More specifically, halotherapy includes heating and crushing the pharmaceutical salt into very tiny micro-particles by a halogenerator creating aerosolization salt. These dry aerosol micro-particles are then blown into the filter fibers where they become trapped in the material. Common standards require that a HEPA air filter must remove—from the air that passes through—at least 99.95% (European Standard)[4] or 99.97% (ASME, U.S. DOE)[5][6] of particles whose diameter is equal to 0.3 μm; with the filtration efficiency increasing for particle diameters both less than and greater than 0.3 μm. Halotherapy will mill and aerosolize the salt particles down to 0.3 to 0.9 microns in diameter, making them small enough to saturate the material of the filter and cover the surface area of the fibers so that when airborne pathogens become trapped in the filter's fibers the salt will absorb the respirated moist air then recrystallize into sharp crystals that will destroy or decode the virus preventing the virus from invading the respiratory tract. The majority of the aerosolization salt particles typically have a diameter of 0.3 to 0.5 microns. The particles of at least 99.99% pure sodium chloride are infused in the non-woven or woven fibers of the filter layer 12. An example of the halogenerator 20 is shown in FIGS. 7 and 8, although larger versions could be developed to scale.

According to one embodiment, the method also includes infusing particles of food grade essential oil into the filter layer 12 along with the at least 99.99% pure sodium chloride particles. In this case, the essential oil can be mixed with the ground particles in the halogenerator 20, and the halogenerator 20 blows both the at least 99.99% pure sodium chloride particles and the essential oil particles into the filter layer 12.

According to one embodiment, a mixture including the pharmaceutical salt, optionally essential oil(s), and optionally other ingredients are infused into the filter layer 12 using the halogenerator 20. The essential oil is a single oil or a blend of oils. The essential oil(s) can include Eucalyptus, Lavender, Tea-tree, Chamomile, Peppermint, Frankincense and/or Vanilla. According to one embodiment, the essential oils include Eucalyptol (high pressure crystalized), Cineole, Mint (crystallized), and Camphor (steam chamber purified and low temperature crystalized).

According to another example embodiment, the filter layer 12 is dipped in a salt bath containing a liquid and a concentration of pharmaceutical salt. This would coat the entire surface area of the first material and all of the fibers.

Once the filter layer 12 containing or infused with the particles of 99.99% pure sodium chloride and optionally the essential oil, the filter layer 12 can be disposed along the facial section 14 of the face mask 10. Typically, the filter includes three layers, so the inner layer 16 is attached to an inner side of the filter layer 12 and the outer layer 18 is attached to an outer side of the filter layer 12. The inner and outer layers 16, 18 can be attached by binding, stamping, sewing, adhesive, or another method.

The filter layer 12 alone or preferably the attached inner, outer, and filter layers 12, 16, 18 together form the filter and are next attached or coupled to the facial section 14 to form the face mask 12. The facial section 14 is designed to cover a human mouth and nose. For example, the dimensions of the facial section 14 could be approximately 8.5 inches in height×20.5 inches in width, but the height and/or the width could vary by +/−2 inches.

The facial section 14 can be formed of any type of material. However, according to preferred embodiments, the facial section 14 is formed of a highly breathable material, for example a mesh or muslin material. The mesh material can be formed of nylon and/or polyester, and includes openings which are visible to the naked eye. For example, the openings can range from 0.1 to 5 mm in diameter, length, and/or width. Forming the facial section 14 from mesh provides for the high breathability, which is especially desirable for individuals participating in physical activities, such as athletes, and wearing the face mask 10 for long periods of time. The prior art face masks which are capable of preventing the transmission of a virus and pathogens are typically formed of a thick cloth fabric and not highly breathable. Such masks do not include openings ranging in size from 0.1 to 5 mm in diameter, length, and/or width, like the mesh facial section. Alternatively, the facial section 14 could be formed of cloth or another fabric. The facial section 14 can also include a patter and/or UV protection.

According to a preferred embodiment, the facial section 14 of the face mask 10 includes a pocket 22, and the filter layer 12 is disposed in the pocket 22. The pocket 22 is typically located on an exterior side of the facial section 14, which is designed to face away from a human nose and mouth. The pocket 22 could also be located in the interior side of the facial section 14. According to another embodiment, the facial section 14 includes two layers of the mesh or other material, and the filter layer 12 is disposed between the two layers. Alternatively, the filter layer 12 could be coupled to the facial section 14 by fasteners, Velcro, adhesive, or another method.

The face mask 10 typically includes straps 24 attached to the facial section 14 for securing the facial section 14 to a human head. For example, the face mask 10 could include a pair of straps 24 extending from side edges of the facial section 14 and extending around the ears or around the entire head. Alternatively, the facial section 14 could be provided in the form of a neck gaiter or another style.

As indicated above, the face mask 10 formed according to the present invention is light in weight, such that an individual wearing the face mask 10 barely feels its presence. In addition, the filter layer 12 provides two-way protection. The pharmaceutical salt in the filter layer 12 prevents the virus or other pathogen from traveling from another person to the individual wearing the mask 10, and it prevents the virus or other pathogen from traveling away from the individual wearing the mask. More specifically, when a virus-carrying droplet encounters the filter layer 12, it starts to absorb the salt, and once the droplets evaporate, all that remains is the virus. The crystallized salt pierces and slices through the virus, neutralizing, deactivating, and decoding it. The process takes about as long as it takes the water droplets to evaporate, very little time. The filter layer 12 will last about 30 days before needing to be replaced with a new one, and the replacement is quick and inexpensive. The facial section 14 of the face mask can be washed and reused.

Obviously, many modifications and variations of the present invention are possible in light of the above teachings and may be practiced otherwise than as specifically described while within the scope of the invention.

1. A method of making a filter, comprising the steps of:
adding particles of at least 99.99% pure sodium chloride to a filter layer.

2. The method of claim 1, wherein the step of adding the particles includes bathing the filter layer in a liquid containing the particles of at least 99.99% pure sodium chloride.

3. The method of claim 1, wherein the filter layer is formed of a carbon material.

4. The method of claim 3, wherein the filter layer contains fibers.

5. The method of claim 1, wherein the particles have a diameter ranging from 0.3 to 0.5 microns.

6. The method of claim 1, wherein the filter layer further includes essential oil.

7. The method of claim 6, wherein the essential oil includes at least one oil selected from the group consisting of Eucalyptus, Lavender, Tea-tree, Chamomile, Peppermint, Frankincense, Vanilla, Eucalyptol, Cineole, Mint, and Camphor.

8. The method of claim 1, wherein the filter layer has a size of 4.7 inch±2 inches×3.1 inch±2 inches.

9. The method of claim 1, wherein the filter layer has a size of 4.8 inches±1 inch×3.86 inches±1 inch.

10. The method of claim 1, wherein the step of adding the particles to the filter layer includes infusing the particles into the filter layer.

11. The method of claim 10, wherein the infusing step is conducted by a halogenerator.

12. The method of claim 1, wherein the filter layer is located between two layers of meltblown cloth, and the filter layer and the two layers of meltblown cloth are located between two layers of SMS spunbonded non-woven material.

13. A method of making a face mask, comprising:
disposing a filter along a facial section material, wherein the filter includes a filter layer containing particles of at least 99.99% pure sodium chloride.

14. The method of claim 13 including forming the filter by bathing the filter layer in a liquid containing the particles of at least 99.99% pure sodium chloride.

15. The method of claim 13, wherein the filter layer is located between two layers of meltblown cloth.

16. The method of claim 15, wherein the filter layer and the two layers of meltblown cloth are located between two layers of SMS spunbonded non-woven material.

17. The method of claim 13, wherein the facial section material includes a pocket, and the filter is disposed in the pocket.

18. The method of claim 13, wherein the facial section material is a mesh material.

19. The method of claim 18, wherein the mesh material is nylon and/or polyester and includes openings ranging from 0.1 to 5 mm.

20. The method of claim 13, wherein the filter layer is located between two layers of meltblown cloth, and the filter layer and the two layers of meltblown cloth are located between two layers of SMS spunbonded non-woven material.
